(19) United States
(12) Patent Application Publication
(10) Pub. No.: US 2006/0191532 A1
(43) Pub. Date: Aug. 31, 2006

(54) Dispensing devices

(76) Inventor: Calvin Ross, Northamptonshire (GB)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/544,698

(22) PCT Filed: Mar. 11, 2004
(86) PCT No.: PCT/GB04/01048

§ 371 Date: Oct. 17, 2005

(30) Foreign Application Priority Data
Mar. 11, 2003 (GB) 0305581.1

(51) Int. Cl. A61M 11/00

(52) U.S. Cl. 128/200.14; 128/200.23

(57) ABSTRACT

The present invention relates to dispensing devices and in particular to spray devices which are used to dispense pharmaceutical compositions in a controlled and safe manner.

The present invention relates to dispensing devices and in particular to spray devices which are used to dispense pharmaceutical compositions in a controlled and safe manner.

A wide variety of pharmaceutical compositions are dispensed as sprays, the compositions being in the form of liquids, that is, solutions or suspensions, gels, or dry powders. These sprayed compositions are provided for use in various modes of administration including inhalation, nasal administration or oral or sublingual administration.

The most widely used type of spray device is probably the metered dose inhaler or MDI which is generally used for administration of pharmaceutical compositions to the lungs, that is, for inhalation. These devices are popular as they are small and portable, allowing the user to carry them around and use when necessary. These devices are also cheap and they tend to be disposable, so that they can simply be discarded when the provided doses have been used.

Whilst the conventional MDIs and other simple spray devices are useful for dispensing some types of pharmaceutical compositions, their use is limited by the fact that there is little or no control over the use of the device. Thus, these conventional types of devices are generally considered to be unsuitable for dispensing drugs which have a narrow therapeutic window so that there is a real risk that the user will overdose without any element of control.

Analgesics having a short duration of action are highly desirable in circumstances where acute severe pain has to be eliminated quickly, for example in cancer sufferers. In view of the potent analgesic activity of such compositions, their use is carefully controlled and monitored. Of particular concern is the risk of inadvertent overdose of such potent compositions, and death can even result due to poor control of dosage regimen.

The management of the delivery of potent substances has been considered over the last ten years and various lock-out mechanisms have been proposed which lock the device after actuation for a predetermined length of time. These devices combine the ability of electronic intelligence with delivery devices such as MDIs and pharmaceutical delivery chambers. A number of devices with lock-out mechanisms are available and they are generally for nasal or lung delivery.

However, the known delivery devices with lock-out mechanisms have the serious disadvantage that they are not disposable. Unlike the conventional MDIs without lock-out mechanisms, the available devices with lock-out mechanisms are expensive, require regular maintenance and calibration and they also tend to be large and bulky. It is usual for known devices with lock-out mechanisms to be delivered, cleaned and maintained by third party providers and the cost is such that these devices are even available for hire.

Thus, according to a first aspect of the present invention, a dispensing device with a lock-out mechanism is provided which is compact and disposable. As soon as the doses held by the device have been dispensed, the device may be discarded.

In a preferred embodiment, the dispensing device comprises a composition to be dispensed, a container for holding the composition, an actuator which is electronically controlled, and a housing within which the various device parts are housed.

The container is preferably made from glass, preferably type 1 pharmaceutical grade, for example as commercially available from Schott Glas (Mainz, Germany) or Bormioli Rocco (Fidenza, Italy). Preferably, the glass container is encased in a protective plastic outer coating. Such coated containers are also readily available from the above mentioned sources.

The lock-out mechanism of the dispensing device according to the present invention is preferably powered by a battery or power cell which forms an integral part of the dispensing device. In a preferred embodiment of the invention, the battery is incorporated within the housing of the device. This can be accomplished by having a bespoke space inside the housing within which the battery fits. Alternatively, the battery may be actually incorporated in the housing, for example, it may be embedded in the housing material, preferably on an inner surface. In a yet further alternative embodiment, the battery is embedded in the plastic coating of the glass container. In another embodiment, the battery is printed on the surface of a plastic part of the device, such as the housing or the coated container.

The batteries used in the devices of the present invention are preferably small in size, so that they do not substantially increase the size and/or weight of the device.

Generally, a 1.5V source of power is required, although the amount of power required will naturally depend upon the size of the doses and the number of doses the device is to dispense. A 1.5V battery will be suitable for dispensing doses of between 25 and 150 μJ in separate doses. Such a battery will also be suitable for a device life of 2-3 years. Larger, more powerful batteries may be used and these are preferably held within the device housing, for example in a bespoke space between the housing and the container.

Suitable battery labels can be used to provide the necessary power for dispensing, say 50 low volume doses of 10 μg per dose.

Other means of powering the dispensing device include mechanical windings which give short bursts of power, fuel storage cells, preferably chemical or nuclear types, and also solar energy. A small solar panel could be attached to the mechanism, either as a primary source of power or as a secondary back-up for recharging a rechargeable battery or the like within the device.

It may also be desirable to have a back-up power source available, for use when the primary power source fails or runs out. An unexpected loss of power would be particularly undesirable if the device is used to dispense emergency and/or life-saving medicine.

In a preferred embodiment, the device is also provided with an indicator of the amount of power left in the device.

The dispensing device will preferably hold between 25 and 50 doses, although devices holding more or fewer doses are also envisaged.

Once the battery is connected to the actuator, the device is ready for use. The power from the battery is used to open or close the actuator, as required.

Where a minimum period of time has to be observed between doses of the composition to be dispensed, the actuator locks after actuation, making it impossible to dispense a further dose until the predetermined time period has elapsed. This is to prevent inadvertent or deliberate overdosing.

Thus, the device will include a control mechanism which is powered by the battery and which controls the action of the actuator. This control mechanism will preferably be an electronic mechanism, and the details of a suitable control mechanism would be known to a skilled person. The control mechanism will be pre-programmed to lock for a given period of time after an actuation.

In a preferred embodiment, the control program will include a counter which monitors the number of doses dispensed by the device, and which displays to the user the number of doses the device has dispensed or the number of doses remaining in the device.

The electronic control mechanism for controlling the actuation of the device is preferably positioned within the device housing. In one embodiment of the invention, the control mechanism is strip printed onto a surface inside the housing of the device. Alternatively, the instructions may be embedded in a plastic moulding.

According to a second aspect of the present invention, a mechanism is provided for preventing inadvertent actuation of the device, for example when the device is stored and particularly when it is being carried around by the user.

In a preferred embodiment of the present invention, a device having a folding actuator or dispensing tube is provided. The dispensing tube moves between a closed position where it is lies folded away, alongside the housing or within a recess in the side of the housing of the device, and an open or active position, where it is positioned at an angle to the surface of the housing, in a position appropriate for comfortable and accurate dispensing the composition to the mouth for sublingual or lung delivery or to the nose for nasal delivery.

A dose of the composition will only be dispensed when the dispensing tube is in the active position. Once a dose has been dispensed, the dispensing tube must be returned to the closed position and then once again put in the active position before a further dose can be dispensed. This will prevent the user from inadvertently dispensing multiple doses.

Where the device includes a lock-out mechanism, the device will only dispense a dose when the dispensing tube is in the active position and when the lock-out mechanism permits. Thus, moving the dispensing tube of the device to the active position is not intended to override the lock-out mechanism.

This device design with the folding dispensing tube means that a separate cap is not required to cover the end of the dispensing tube. This is more convenient, as such caps are easily lost or they can fall off, especially when the device is being carried around by the user.

Thus, it is clear that the device according to the present invention above is simple, cheap and disposable.

It is frequently desirable to ensure that the container carrying the pharmaceutical composition to be dispensed cannot be removed from the housing of the device.

Therefore, the device preferably has a locking mechanism, such as a ratchet, which allows the container to be easily inserted into the housing but will not permit its removal. In an alterative embodiment, the housing comprises parts which are permanently attached to one another once the container and other device parts have been inserted, thereby encasing the container and making it impossible to remove it or tamper with it. The housing parts may be glued together or, more preferably, they are welded together, for example by ultrasonic welding.

The device of the present invention is particularly suitable for dispensing pharmaceutical compositions such as potent the pain-relieving agents fentanyl and other opioid analgesics, such as alfentanil, carfentanil, remifentanil, sufentanil, buprenorphine, morphine, diamorphine, and the like. Opioid analgesics are frequently used for the relief of moderate to severe pain and the rapid onset of their effect makes them ideal candidates for treating break-through pain. However, the use of such opioid analgesics is currently restricted by the need to tightly control dosing to prevent overdosing or abuse.

The devices of the present invention are particularly suitable for patient-controlled analgesia, where the patient self-administers the drug in response to the pain he or she is experiencing. In order for such patient-controlled pain management to be effective, the drugs must be available to the patient in such a way that they can be easily and safely administered. In particular, it is important that the patient does not overdose. Large doses of these drugs may lead to respiratory depression and some euphoric activity which can lead to abuse and dependency. The side-effect of respiratory depression is another reason why self-dosing is attractive for these drugs. Self-administration allows the patient to adjust the frequency of doses according to the respiratory depression experienced.

The devices according to the present invention may include any known type of valve, for example inverted valves or upright valves. Devices with inverted valves are generally preferred, as the volumes of pharmaceutical composition that they dispense are more accurate and consistent. Dispensing an accurate dose is of particular importance when administering such powerful drugs as opioid analgesics.

Examples of suitable upright valve devices for dispensing the pharmaceutical compositions described herein are readily available from Bespak PLC of Bergen Way, Kings Lynn, Norfolk PE30 2JJ, United Kingdom. Devices with upright valves include those disclosed in WO 92/11190, U.S. Pat. No. 4,819,834 and U.S. Pat. No. 4,407,481.

An example of a device according to the present invention will now be described, by way of example only, and with reference to the following drawings.

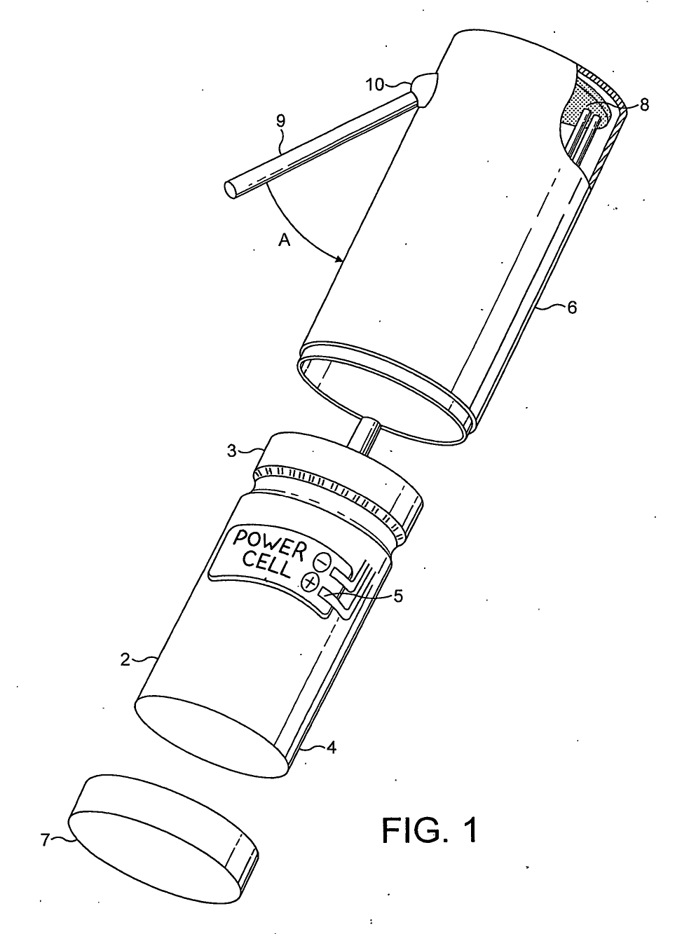

FIG. 1 is an exploded view of an embodiment of a device in accordance with the invention, intended for sublingual administration of a pharmaceutical composition.

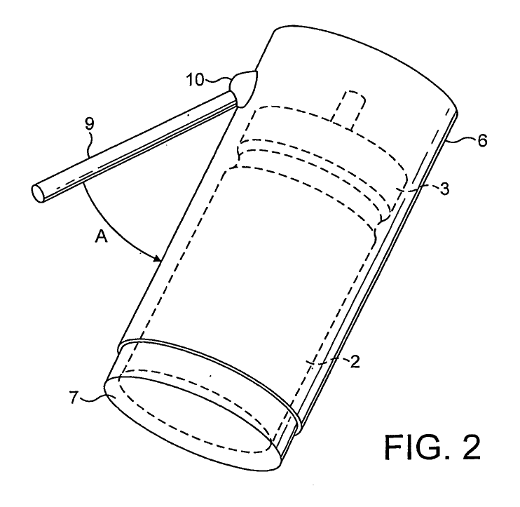

FIG. 2 shows the assembled view of the device of FIG. 1.

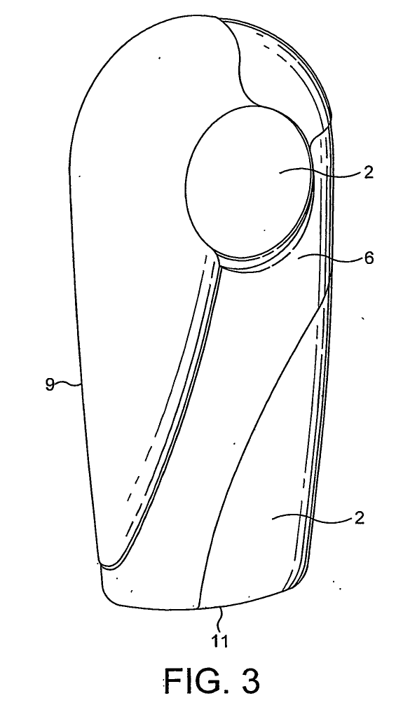

FIG. 3 shows an example of a device in accordance with the invention with the actuator in the closed position.

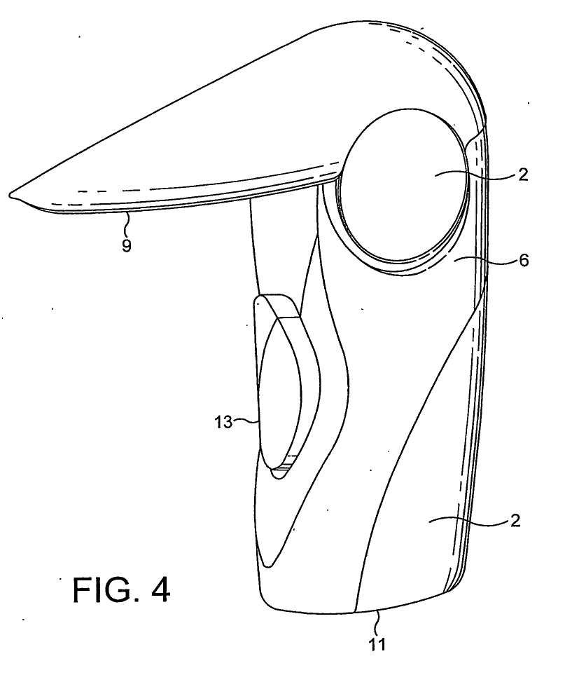

FIG. 4 shows the device of FIG. 3, with the actuator in the active position.

The device shown in FIGS. 1 and 2 comprises a substantially cylindrical container 2 sealed with a cap 3. Both the container 2 and the cap 3 may be manufactured from a variety of materials. The container and cap may be lined with a polyester (such as Celanex® 2500) or a lacquer (not shown). The glass container 2 has a moulded plastic coating 4 on its outer surface.

The container 2 is encased in a housing comprising a housing body 6 and a housing cap 7. This two-part housing configuration allows the container 2, and other device parts, to be placed within the housing and then sealed in.

Printed on or embedded in the plastic coating 4, there is a power cell or battery 5. In an alternative embodiment which is not shown, the power cell or battery may be positioned on the inside surface of the housing body 6 or the housing cap 7. In yet another embodiment which is not shown, a more conventional battery, such as one used in watches and hearing aids, may be fitted inside the housing cap 7, in a space between the housing cap and the end of the container 2.

The electronic control mechanism 8, which is powered by the battery or power cell 5, is positioned on the inner surface of the housing body 6.

The device is provided with a dispensing tube or actuator 9, through which the composition passes when it is dispensed. This tube 9 forms a channel between the container 2 and the device exterior and it is this tube which must be correctly positioned in the user's mouth when the device is used.

Arrow A shows the range of movement of the dispensing tube 9. The tube moves between a closed position where it lies alongside the housing body 6 to an open or active position, which is illustrated. Naturally, the exact orientation of the active position will depend upon the intended mode of administration, the orientation being selected for easy and comfortable use.

The actuator or dispensing tube 9 is moved by virtue of a flexible hinge 10. Preferably, the hinge has two pre-set positions, so that the dispensing tube is either in the closed position or the active position. Preferably, the device can sense whether or not the dispensing tube is in the open position and therefore whether or not a dose can be dispensed.

The device shown in FIGS. 3 and 4 is a more refined version of a device. The device comprises a container 2 within the housing body 6. A window 11 in the housing body allows the container and its contents to be viewed from outside the housing, allowing the user to see when the formulation stored within the container is running low.

The device has an actuator 9 which is movable between the closed position shown in FIG. 3 and the active position in FIG. 4. The device can only dispense the formulation when the actuator 9 is in the active position shown in FIG. 4. Once again, the actuator moves about a hinge 12 which locks at two positions, the closed and the active positions.

This is reinforced by the fact that the button 13 which allows the device to dispense a dose of the pharmaceutical formulation is hidden when the actuator 9 is in the closed position and it cannot be seen or activated until the actuator is moved into the active position.

1. A device for dispensing a pharmaceutical composition comprising a composition to be dispensed, a container for holding the composition, an actuator which is electronically controlled, and a housing within which the various device parts are housed.

2. The device as claimed in claim 1, wherein the device is compact and disposable.

3. The device as claimed in claim 1, wherein the container is made from glass, preferably type 1 pharmaceutical grade glass.

4. The device as claimed in claim 3, wherein the glass container is encased in a protective plastic outer coating.

5. The device as claimed in claim 1, wherein the device further comprises a lock-out mechanism which is compact and disposable.

6. The device as claimed in claim 5, wherein the lock-out mechanism is powered by a battery or power cell which forms an integral part of the dispensing device.

7. The device as claimed in claim 6, wherein the battery or power cell is incorporated within the housing of the device.

8. The device as claimed in claim 6, wherein the battery or power cell is incorporated in the housing.

9. The device as claimed in claim 6, wherein the battery or power cell is embedded in a plastic coating surrounding the glass container.

10. The device as claimed in claim 6, wherein the battery or power cell is printed on the surface of a plastic part of the device, such as the housing or the plastic coated container.

11. The device as claimed in claim 6, wherein the battery or power cell provides a 1.5V source of power.

12. The device as claimed in claim 1, wherein the device includes a power source comprising mechanical windings, fuel storage cells, preferably chemical or nuclear types, or solar energy.

13. The device as claimed in claim 1, wherein the device includes a back-up power source.

14. The device as claimed in claim 1, wherein the device further comprises an indicator of the amount of power left in the device.

15. The device as claimed in claim 1, wherein power from a battery or power cell is used to open or close the actuator.

16. The device as claimed in claim 1, wherein the device comprises a control mechanism which is powered by a battery and which controls the action of the actuator.

17. The device as claimed in claim 16, wherein the control mechanism is an electronic mechanism.

18. The device as claimed in claim 16, wherein the control mechanism is pre-programmed to lock for a given period of time after an actuation.

19. The device as claimed in claim 1, wherein the device includes a counter which monitors the number of doses dispensed by the device.

20. The device as claimed in claim 17, wherein the electronic control mechanism is positioned within the device housing.

21. The device as claimed in claim 20, wherein the electronic control mechanism is strip printed onto a surface inside the housing of the device.

22. The device as claimed in claim 20, wherein the electronic control mechanism is embedded in a plastic moulding forming part of the device.

23. The device as claimed in claim 1, wherein the device has a folding actuator or dispensing tube which moves between a closed position where it lies folded away, alongside the housing or within a recess in the side of the housing of the device, and an open or active position, where it is positioned at an angle to the surface of the housing, in a position appropriate for comfortable and accurate dispensing the composition to the mouth for sublingual or lung delivery or to the nose for nasal delivery.

24. The device as claimed in claim 23, wherein the composition can only be dispensed when the actuator or dispensing tube is in the active position.

25. The device as claimed in claim 23, wherein the actuator or dispensing tube must be returned to the closed position following the dispensing of a dose of the pharmaceutical composition and then moved back to the active position before a further dose can be dispensed.

26. The device as claimed in claim 23, wherein the device includes a lock-out mechanism and the actuator or dispensing tube can only be moved into the active position when the lock-out mechanism permits.

27. The device as claimed in claim 1, wherein the container holding the pharmaceutical composition cannot be removed from the housing of the device.

28. The device as claimed in claim 27, wherein a ratchet prevents removal of the container from the housing.

29. The device as claimed in claim 27, wherein the housing comprises parts which are permanently attached to one another once the container and other device parts have been inserted, thereby preventing removal of the container from the housing.

30. The device as claimed in claim 29, wherein the housing parts are permanently attached to one another by glue or they are welded together, for example by ultrasonic welding.

31. The device as claimed in claim 1, wherein the pharmaceutical composition comprises an opioid analgesic.
